(19) United States
(12) Patent Application Publication
(10) Pub. No.: US 2016/0045553 A1
(43) Pub. Date: Feb. 18, 2016

(54) CELL DELIVERY

(71) Applicant: THE UNIVERSITY OF BIRMINGHAM, Birmingham, West Midlands (GB)

(72) Inventors: Liam GROVER, West Midlands (GB); Fotios SPYROPOULOS, West Midlands (GB); Jennifer PAXTON, West Midlands (GB)

(21) Appl. No.: 14/775,441

(22) PCT Filed: Mar. 10, 2014
(86) PCT No.: PCT/GB2014/050708

§ 371 Date: Sep. 11, 2015

(30) Foreign Application Priority Data
Mar. 13, 2013 (GB) 1304514.1

(51) Int. Cl. A61K 35/32; A61L 2/08; A61L 2/10; A61K 9/14

(52) U.S. Cl. CPC A61K 35/32; A61K 9/146; A61L 2/081; A61L 2/082; A61L 2/10

(57) ABSTRACT

The invention provides a cell delivery medium comprising a liquid phase, wherein the liquid phase comprises (i) one or more cells suspended within the liquid phase and (ii) a plurality of polymer gel particulates. Methods of producing the cell delivery systems are also provided.

A cell delivery medium according to the invention for use in the treatment of diseased or damaged tissue.

The use of cross-linked gels containing cells has been known for a number of years. For example, using cells trapped within a gel matrix. For example, US2005/0003010A describes a cross-linked alginate obtained by cross-linking sodium alginate solution by the addition of calcium ions to form a gel. Such gels are used to induce cell proliferation, by, for example, injecting the solution into damaged tissue. The cross-linking is broken on shearing, for example by passing through a needle to a site to be treated.

US2007/0116680 describes embedding stem cells in a three dimensional hydrogel. Stem cells are suspended in a matrix solution, the matrix is gelled and the cells are contained within microbeads formed from the matrix.

Hydrogel encapsulated stem cells are also disclosed in US2012/0027860A. Adipose-derived mesenchymal stem cells are mixed with a gel forming solution prior to causing the solution to gel.

The applicant has identified that producing cell delivery medium in which cells are suspended within a liquid phase containing polymer fluid gel microparticles, but not embedded within these, allows media with advantageous properties to be produced.

The rheological properties of such fluid gels “liquid phases” can be controlled, for example by the methods of the invention, to allow these systems to have a range of flow functionalities when applied at a desired location where they should be retained. Properties of the fluid gels can be tailored to each specific application (to be injectable, spreadable, shear-thinning, Newtonian, etc) by controlling the formulation and processing parameters involved during their manufacture.

For example, a product containing a patient's own cells could be spread over a dermal wound or ulcer to expedite healing. Or, autologous cells could be localised to an area of musculoskeletal damage or disease by injection e.g. around or into a damaged tendon. The fluid gel structure itself as well as its rheological properties can be also carefully formulated, if required by specific applications for these systems, to be transient rather than stable. For example, a fluid gel structure can be designed to revert to a typical (non-sheared) gel structure as a function of time, external stimuli (temperature, ionic charge, etc.). This can allow for the formulation of a fluid gel structure carrying therapeutic cells that is initially injectable but subsequently, e.g. after application at the point of injury, to form a structure that is firmly anchored but has been shaped to occupy the available space in the body (e.g. not causing any disruption to movement) and is still retaining the active cells. Similarly the actual flow behaviour of the fluid gels (even when these do not undergo any structural transformation such as that described above) can be designed to be transient. This can allow for specific flow characteristics (e.g. a high yield stress, etc.) initially exhibited by the system to be either irreversibly or reversibly altered following application; for example, in the case of a system exhibiting a high yield stress, this behaviour can be temporarily eliminated by application of shear (while it is injected) during application, but once shear is removed the yield stress functionality returns after a certain (and again controllable) “resting period”.

The invention provides, a cell delivery medium comprising a liquid phase, wherein the liquid phase comprises (i) one or more cells suspended (or entrapped) within the liquid phase and (ii) a plurality of polymer gel particles. That is, typically the cells are not entrapped within the particles but are within the liquid phase. Mixtures of cells within both the liquid phase and particles may also be provided. Different cells may be used in each of the liquid phases and particles. The liquid phase typically comprises a cell growth medium for said cells.

A further aspect of the invention provides a cell delivery medium comprising a cell growth medium and a plurality of polymer gel particles, wherein said polymer gel particles do not encapsulate one or more cells.

The liquid phase may be any suitable liquid, especially aqueous liquid for suspending viable cells. Typically it is a cell growth medium

The cell growth medium may be any suitable cell growth media depending on the cells to be added. Typically, cell growth media contain a source of amino acids and nitrogen, a carbon source, such as glucose, water and/or a number of different salts needed for cell growth.

Cell growth media may be a growth media for prokaryotic, such as bacterial growth, or eukaryotic growth media. Examples of bacterial growth media includes those utilising a beef or yeast extract, and include selected media such as MacConkey, YM (yeast and mould) and mannitol salt agar.

More typically, the growth media will be suitable for eukaryotic cell growth.

Typical mammalian cell culture media includes Dulbecco's, Ham's, minimum essential medium (MEM), and RPMI-1640. Such media are generally well-known in the art. They may be supplemented with, for example, serum, for example fetal bovine serum (FBS).

The cells may be prokaryotic or eukaryotic. Typically the cells are plant or animal cells, for example, bird, inspect, reptile, more typically mammal cells.

Stem cells may be used. Stem cells are typically human or non-human, pluripotent or totipotent, typically not human totipotent stem cells. The stem cells may be obtained from cell banks or, for example, embryonic, non-embryonic (typically non-embryonic human stem cells), cord blood stem cells or adult mesenchymal stem cells. They may be obtained from single blastomere biopsy, a non-destructive method of producing embryonic stem cells, or from adult cells such as iPS (induced pluripotent stem) cells. Other cells such as differentiated cell lines, or cells isolated from the blood or tissue may be used. The cells may be a patient's own cells. They may be autologous cells.

They may be osteoblasts/MC 3T3 osteoblast like cells, chondrocytes, keratinocytes, fibroblasts, dermal fibroblasts, tenocytes, neurons, osteocytes, osteoclasts, adipocytes or any other cell type with therapeutic activity.

The polymer gel may be selected from agarose, agar, carrageenan, gellan gum, gelatin, pectin, alginate and fibrin. Non-naturally occurring gels, for example, polyacrylate and polyethylene glycol, may be used. Other suitable gels include chitosan, dextran, collagen and hyaluronic acid.

The particles may be substantially spherical, needle or threadlike. The particles may be within substantially a single size distribution family or within several discrete size distribution features.

Typically the average size of the polymer gel particles is 1 to 1000 μm, 1 μm to 500 μm or 10 to 100 μm, or 30 to 50 μm.

The cell delivery medium, according to the invention, may utilise in the polymer gel and/or liquid phase, one or more additional nutrients, antibiotics, hormones, growth factors, inflammatory compounds, cell stimulating factors or other compounds useful in maintaining the cells within the cell delivery medium, encouraging the cells where appropriate to differentiate or for treating the site where the cells are administered in a patient. The antibiotics, for example, may be used to ensure that the cell delivery medium remains substantially bacteria free. Alternatively, the antibiotics may also be used to treat an infection at a site to be treated.

The invention accordingly also includes within the scope pipettes or syringes comprising cell delivery medium according to the invention.

A further aspect of the invention provides a cell delivery medium according to the invention for use in the treatment of diseased or damaged tissue. Methods of treatment using cell delivery medium are also provided.

A further aspect of the invention provides a method of producing a cell delivery medium comprising:
Dissolving a gelling polymer gel in a liquid phase to form a mixture existing in a liquid state;
inducing gelation of the polymer gel liquid mixture under application of shear to form a mixture comprising a plurality of polymer gel fluid gel particulates within a liquid phase; and
adding one or more cells to form a suspension of the cells within the liquid phase.

The invention also provides a method of producing a cell suspension medium comprising:
(i) heating a polymer gel in a growth medium liquid phase to above the melting temperature of the polymer gel to form a heated mixture; and
(ii) cooling the heated mixture under shearing to form a mixture comprising a plurality of polymer particulates within the liquid phase.

Cell delivery systems obtainable or made by the methods of the invention are also provided.

Cells may be provided in one or both of the liquid phase and particulates. The cells may be the same or different.

At least the liquid phase may comprise a cell growth medium. A still further aspect of the invention provides a method of producing a cell delivery medium comprising:

(i) heating a polymer gel in a growth medium liquid phase to above the melting temperature of the polymer gel to form a heated mixture; and

(ii) cooling the heated mixture under shearing to form a mixture comprising a plurality of polymer gel microparticles within the liquid phase.

The cells, polymer gel, liquid phase in growth medium may be as defined above.

The mixture comprising the plurality of polymer gel microparticles may be sterilised by irradiation, typically prior to addition of the cells. Irradiation may utilise, for example, ultra-violet, x-ray or gamma ray radiation to sterilise the medium, for example, to remove unwanted bacterial contamination.

Typically, the shearing is induced by passing the heated mixture through a pin stirrer as it is cooled. For example, the media may be passed through a water bar to cool the cells that are not encapsulated within a polymer gel matrix, but are suspended within the liquid phase of a fluid gel system and surrounded by individual fluid gel microparticles, fluid gels with advantageous properties can be produced. This allows, for example, the cells to be mobile within a flowable, spreadable or injectable solution depending on the processing characteristics of the fluid gel component. Indeed, the properties of the fluid gels can be tailored for each specific application by controlling the formulation and processing parameters involved during the manufacture. This means that there is the ability to specifically design the functionality of the cell carrying system for use in different scenarios and applications. A further advantage of such a fluid gel system is that while the cells remain viable within the liquid phase of the fluid gel, they also have a lower distance for nutrients and metabolic products to travel (compared to the corresponding situation in a solid gel monolith). Thus, cell viability is likely to remain high at the time of, and following, application at the desired site.

The fluid gel structures are described in the invention below, as the production of thixotropic gels become liquid when a shear force is applied, but the regain a gel-like consistency once the shear force is removed.

The invention will now be described by way of example only, with reference to the following figures:—

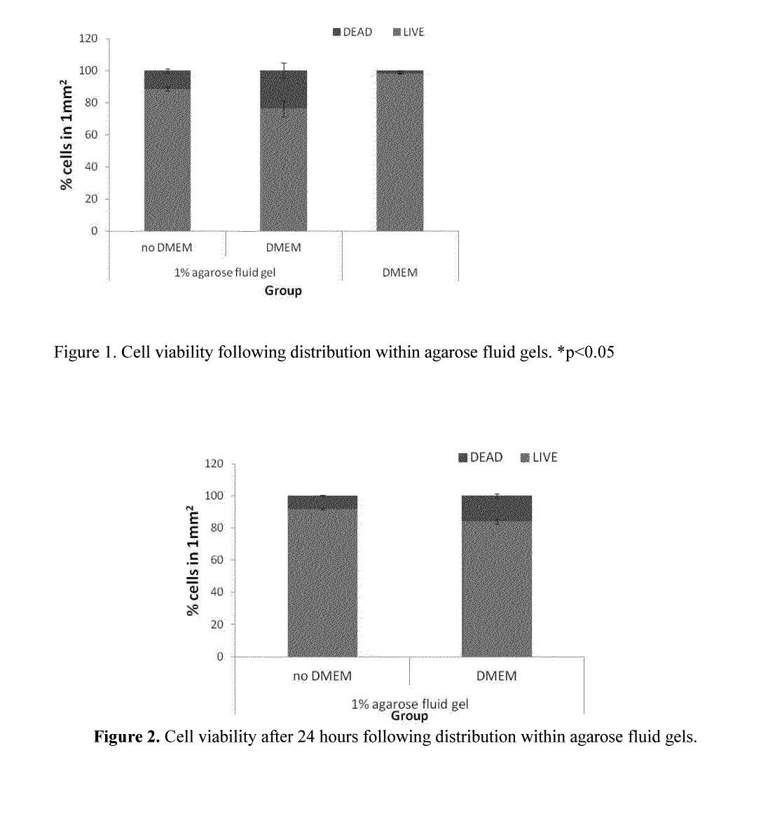

FIG. 1 shows the viability of agarose gels prepared according to the invention, using cells prior suspended in DMEM or cells suspended directly into the gel.

FIG. 2 shows cell viability 24 hours after distribution for directly suspended cells (no DMEM), or via prior delivery in DMEM (DMEM). Example: Agarose fluid gels for cell delivery

A 1% agarose solution was produced by dissolving agarose powder in Dulbecco's Modified Eagles Medium (DMEM) under constant agitation at a temperature of approximately 90° C. The agarose solution was kept above its melting temperature for at least 30 minutes before the manufacture of the fluid gel. Agarose fluid gels were produced by subjecting the agarose solution to a shear rate of 1345 rpm whilst cooling using a pin stirrer. The temperature decrease used to induce gelation of the hydrocolloid solution was provided by means of a cooling jacket, surrounding the pin-stirrer, maintained at a constant temperature of 25° C. by a circulating water bath. Agarose solution was pumped through the processing apparatus at a flow rate of 10 ml/min. Following fluid gel production, the fluid gel was sterilised by UV light irradiation for 20 minutes in a laminar flow cabinet.

MC-3T3 cells were cultured and passaged routinely in supplemented DMEM (s-DMEM), containing 10% fetal bovine serum (FBS), 1% penicillin/streptomycin, 2.4% L-glutamine and 2.4% HEPES buffer until required. To produce a cell-associated fluid gel, MC-3T3 cells were detached from the polystyrene flask surface using TryPLe and centrifuged at 1000 rpm for 3 minutes. Following centrifugation, cells were added to the sterile fluid gel by one of two methods;

1) The cell pellet was resuspended in 500 ρl of s-DMEM and then combined with agarose fluid gel. The cell pellet was directly resuspended in agarose fluid gel. In both cases the cells were distributed throughout the fluid gel by pipette mixing and had a final cell concentration of 500,000 cells per ml of final solution volume. 1 ml samples of cell-associated fluid gel were placed in the wells of a 24 well plate. 1 ml of s-DMEM was added to each well and the samples were incubated at 37° C., 5% CO2.

Once the cell-associated fluid gel was produced, the viability of the cells was measured to assess whether distributing cells within the fluid phase of the fluid gel would be detrimental to cell viability. Samples were incubated with Calcein-AM/Propidium iodide at concentrations of 0.1 μg/ml and 2 μg/ml respectively for 30 minutes before being viewed by fluorescence microscopy. This allowed a simultaneous view of live (lower band) and dead (upper band) cells. Multiple photographs were taken of different fields of view, and these were used to calculate the percentage live/dead cells in a defined area of each sample (1 mm2). Mixing the cell population with agarose fluid gel does not have a significant effect on cell viability, when comparing samples made by method 1 or method 2 (p=0.12). Fluid gel samples manufactured using additional DMEM displayed a significant drop in cell viability when compared to cells that were suspended in s-DMEM alone (p=0.03) (FIG. 1), however, viability still remained high, at approximately 80%.

To investigate cell viability over time, samples were incubated in s-DMEM for 24 hours at 37° C., 5% CO2 and stained as described above. Cell viability remained high, at around 90%, with no significant difference between samples (p=0.35) (FIG. 2).

1. A cell delivery medium comprising a liquid phase, wherein the liquid phase comprises (i) one or more cells suspended within the liquid phase and (ii) a plurality of polymer gel particulates.

2. A cell delivery system according to claim 1, for use in the treatment of diseased or damaged tissue.

3. A cell delivery medium according to claim 1, wherein the liquid phase and/or at least a portion of the polymer gel particulates comprises a cell growth medium for said cells.

4. A cell delivery medium according to claim 1, wherein substantially only the liquid phase contains one or more cells and the polymer gel particulates do not enclose substantially any cells.

5. A cell delivery medium according to claim 1, wherein the liquid phase contains one or more cells and the polymer gel particulates enclose one or more cells, with said cells being of the same type as the cells in the liquid phase.

6. A cell delivery medium according to claim 1, wherein the liquid phase contains one or more cells and the polymer gel particulates enclose one or more cells, said cells being of different type to the cells in the liquid phase.

7. A cell delivery medium according to claim 1, wherein the polymer gel is selected from agarose, agar, carrageenan, gellan, gelatin, pectin, alginate or fibrin gels.

8. A cell delivery medium according to claim 1, wherein the average size of the polymer gel particulates is 1 μm to 500 μm.

9. A cell delivery medium according to claim 1, wherein the polymer gel and/or liquid phase comprises one or more nutrients, antibiotics, hormones, growth factor, anti-inflammatory compounds and/or cell stimulating factors.

10. A cell delivery medium according to claim 1, comprising one or more cells suspended in the liquid phase, and/or the same or different cells within the polymer gel particulates are selected from stem cells or differentiated cells.

11. A pipette or syringe comprising a cell delivery medium according to claim 1.

12. A method of producing a cell delivery medium comprising:
Dissolving a gelling polymer gel in a liquid phase to form a mixture existing in a liquid state;
inducing gelation of the polymer gel liquid mixture under application of shear to form a mixture comprising a plurality of polymer gel fluid gel particulates within a liquid phase; and
adding one or more cells to form a suspension of the cells within the liquid phase.

13. A method of producing a cell delivery medium comprising:
Dissolving a gelling polymer gel in a liquid phase to form a mixture existing in a liquid state;
adding one or more cells to the polymer gel liquid mixture; and
inducing gelation of the cell-containing polymer gel liquid mixture under application of shear to form a mixture comprising a plurality of polymer gel particulates within a liquid phase, with the said particulates enclosing the majority of the added one or more cells.

14. A method of producing a cell delivery medium according to claim 13 comprising:
dissolving a gelling polymer gel in a liquid phase to form a mixture existing in a liquid state;
adding one or more cells to the polymer gel liquid mixture;
inducing gelation of the cell-containing polymer gel liquid mixture under application of shear to form a mixture comprising a plurality of polymer gel fluid gel particulates within a liquid phase, with the said particulates enclosing the majority of the cells; and
adding one or more further cells, with said cells being of the same type as cells, liquid phase or of a different type to the liquid phase cells to form a suspension of the cells within the liquid phase of the cell delivery medium.

15. A method according to claim 12, wherein gelation of the biopolymer liquid mixture is induced by:
(i) cooling the said mixture below the melting temperature of the gelling biopolymer; or
(ii) adding ionic species, or mixtures of ionic species, to the said biopolymer mixture; or
(iii) acidifying the said mixture to the required pH conditions.

16. A method according to claim 12, wherein at least the liquid phase comprises a cell growth medium.

17. A method of producing a cell suspension medium comprising:
(i) heating a polymer gel in a growth medium liquid phase to above the melting temperature of the polymer gel to form a heated mixture; and
(ii) cooling the heated mixture under shearing to form a mixture comprising a plurality of polymer gel particulates within the liquid phase.

18. A method according to claim 12, wherein the mixture comprising the plurality of polymer gel particulates is sterilised by irradiation.

19. (canceled)

20. A method according to claim 12, wherein said polymer gel particles do not encapsulate one or more cells.

21. A method according to claim 12, wherein the polymer gel is selected from agarose, agar, carrageenan, gellan, gelatin or fibrin gels.

22. A method according to claim 12, wherein the average size of the polymer gel particles is 1 μm to 500 μm.

23. A cell delivery system obtained by a method according to claim 12.

24. A method according to claim 13 wherein gelation of the biopolymer liquid mixture is induced by:
(i) cooling the said mixture below the melting temperature of the gelling biopolymer; or
(ii) adding ionic species, or mixtures of ionic species, to the said biopolymer mixture; or
(iii) acidifying the said mixture to the required pH conditions.

25. A method according to claim 13 wherein at least the liquid phase comprises a cell growth medium.

26. A method according to claim 13, wherein said polymer gel particles do not encapsulate one or more cells.

27. A method according to claim 13, wherein the polymer gel is selected from agarose, agar, carrageenan, gellan, gelatin or fibrin gels.

28. A method according to claim 13, wherein the average size of the polymer gel particles is 1 μm to 500 μm.

29. A cell delivery system obtained by a method according to claim 13.
